(19) United States
(12) Patent Application Publication
(10) Pub. No.: US 2005/0008540 A1
(43) Pub. Date: Jan. 13, 2005

(54) Biochemical analysis unit structure body

(75) Inventors: Masanori Ishiguro, Odawara-shi (JP); Kenji Nakajima, Minamiashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/860,567

(22) Filed: Jun. 4, 2004

(30) Foreign Application Priority Data
Jun. 6, 2003 (JP) 162107/2003

(51) Int. Cl. G01N 33/00

(52) U.S. Cl. 422/81

(57) ABSTRACT

A biochemical analysis unit structure body comprises a housing, which is provided with a support section for supporting a biochemical analysis unit within the housing, the biochemical analysis unit comprising a base plate, which has a plurality of holes, and a porous adsorptive material, which is filled in each of the plurality of the holes of the base plate and forms each of a plurality of adsorptive regions. A flow path is formed in the housing and enables a liquid to be circulated through each of the adsorptive regions of the biochemical analysis unit. A flowing member is located in the flow path and circulates the liquid through the flow path by being actuated by actuating device.

### BACKGROUND OF THE INVENTION

1. Field of the Invention

This invention relates to a biochemical analysis unit structure body, which is adapted for use in an operation for detecting a receptor or a ligand by the utilization of a labeling substance.

2. Description of the Related Art

Various micro array analysis systems and various macro array analysis systems have heretofore been used. With the micro array analysis systems and the macro array analysis systems, liquids containing ligands or receptors (i.e., the substances, which are capable of specifically binding to organism-originating substances and whose base sequences, base lengths, compositions, characteristics, and the like, are known) are spotted onto different positions on a surface of a biochemical analysis unit, such as a membrane filter, and a plurality of adsorptive regions are thereby formed on the surface of the biochemical analysis unit. Examples of the ligands or the receptors include hormones, tumor markers, enzymes, antibodies, antigens, abzymes, other proteins, nucleic acids, cDNA's, DNA's, and RNA's. Thereafter, a labeled receptor or a labeled ligand, which has been labeled with a radioactive labeling substance, a fluorescent labeling substance, a labeling substance capable of causing a chemical luminescence substrate to produce chemical luminescence when being brought into contact with the chemical luminescence substrate, or the like, is subjected to hybridization, or the like, with the ligands or the receptors, which are contained in the adsorptive regions of the biochemical analysis unit. The labeled receptor or the labeled ligand is thus specifically bound to at least one of the ligands or the receptors, which are contained in the adsorptive regions of the biochemical analysis unit. The labeled receptor or the labeled ligand is the substance, which has been sampled from an organism through extraction, isolation, or the like, or has been subjected to chemical treatment after being sampled, and which has been labeled with the radioactive labeling substance, the fluorescent labeling substance, the labeling substance capable of causing a chemical luminescence substrate to produce the chemical luminescence when being brought into contact with the chemical luminescence substrate, or the like. Examples of the labeled receptors or the labeled ligands include hormones, tumor markers, enzymes, antibodies, antigens, abzymes, other proteins, nucleic acids, DNA's, and mRNA's.

In cases where the labeled receptor or the labeled ligand has been labeled with the radioactive labeling substance, a stimulable phosphor layer of a stimulable phosphor sheet is then exposed to radiation radiated out from the radioactive labeling substance, which is contained selectively in the adsorptive regions of the biochemical analysis unit. Thereafter, the stimulable phosphor layer is exposed to stimulating rays, which cause the stimulable phosphor layer to emit light in proportion to the amount of energy stored on the stimulable phosphor layer during the exposure of the stimulable phosphor layer to the radiation. The light emitted by the stimulable phosphor layer is detected photoelectrically, and data for a biochemical analysis is thereby obtained.

In cases where the labeled receptor or the labeled ligand has been labeled with the fluorescent labeling substance, excitation light is irradiated to the adsorptive regions of the biochemical analysis unit, and the fluorescent labeling substance, which is contained selectively in the adsorptive regions of the biochemical analysis unit, is excited by the excitation light to produce fluorescence. The thus produced fluorescence is detected photoelectrically, and data for a biochemical analysis is thereby obtained.

In cases where the labeled receptor or the labeled ligand has been labeled with the labeling substance capable of causing a chemical luminescence substrate to produce the chemical luminescence when being brought into contact with the chemical luminescence substrate, the labeling substance, which is contained selectively in the adsorptive regions of the biochemical analysis unit, is brought into contact with the chemical luminescence substrate. Also, the chemical luminescence produced by the labeling substance is detected photoelectrically, and data for a biochemical analysis is thereby obtained.

The micro array analysis systems and the macro array analysis systems are described in, for example, U.S. Patent Laid-Open No. 20020016009.

With the micro array analysis systems and the macro array analysis systems described above, a large number of the adsorptive regions, to which the ligands or the receptors are bound, are capable of being formed at a high density at different positions on the surface of the biochemical analysis unit, and the labeled receptor or the labeled ligand, which has been labeled with the labeling substance, is capable of being subjected to the hybridization, or the like, with the ligands or the receptors, which have been bound to the adsorptive regions formed at a high density at different positions on the surface of the biochemical analysis unit. Therefore, the micro array analysis systems and the macro array analysis systems described above have the advantages in that a receptor or a ligand is capable of being analyzed quickly.

Heretofore, with the biochemical analysis systems using a biochemical analysis unit, the hybridization, or the like, has ordinarily been performed with a shaking technique. With the shaking technique, the biochemical analysis unit, on which the ligands or the receptors have been fixed, is put into a hybridization bag, and a reaction liquid, which contains the labeled receptor or the labeled ligand, is added into the hybridization bag. Also, vibrations are given to the hybridization bag, and the labeled receptor or the labeled ligand is thus moved through convection or diffusion within the hybridization bad. In this manner, the labeled receptor or the labeled ligand is specifically bound to at least one of the ligands or the receptors having been fixed on the biochemical analysis unit.

However, with the shaking technique described above, it is not always possible to achieve uniform contact of the reaction liquid containing the labeled receptor or the labeled ligand with the plurality of the adsorptive regions, to which the ligands or the receptors have been fixed. Therefore, the problems occur in that the ligands or the receptors and the labeled receptor or the labeled ligand cannot efficiently be subjected to the binding. In order to solve the problems described above, the applicant proposed a technique, wherein a reaction liquid containing a labeled receptor or a labeled ligand is forcibly caused to flow across each of adsorptive regions of a biochemical analysis unit, such that the labeled receptor or the labeled ligand may penetrate sufficiently into the interior of each of the adsorptive regions of the biochemical analysis unit.

However, such that the reaction liquid containing the receptor or the ligand to be analyzed is capable of being forcibly caused to flow across each of the adsorptive regions of the biochemical analysis unit, and such that the receptor or the ligand is capable of penetrating sufficiently and uniformly into the interior of each of the adsorptive regions of the biochemical analysis unit, it is necessary for a certain amount of the reaction liquid to be utilized. Therefore, in cases where the amount of a sample of the receptor or the ligand to be analyzed, which sample is available, is limited, the problems occur in that the concentration of the sample in the reaction liquid becomes low, and the sensitivity becomes low. Also, ordinarily, in order for accurate data to be obtained from the biochemical analysis, it is important to prevent the problems with regard to contamination by foreign substances from occurring.

### SUMMARY OF THE INVENTION

The primary object of the present invention is to provide a biochemical analysis unit structure body, wherein a receptor or a ligand to be analyzed is capable of being caused to penetrate sufficiently and uniformly into the interior of each of adsorptive regions of a biochemical analysis unit in cases where the amount of a reaction liquid is small.

Another object of the present invention is to provide a biochemical analysis unit structure body, wherein problems with regard to contamination by foreign substances are capable of being prevented from occurring.

A further object of the present invention is to provide a biochemical analysis unit structure body, wherein a reaction is capable of being performed in a state in which a concentration of a sample is high, and a reaction rate is thus capable of being kept high.

The specific object of the present invention is to provide a biochemical analysis unit structure body, wherein a biochemical analysis unit is capable of being prevented from undergoing deflection or deformation.

The present invention provides a biochemical analysis unit structure body, comprising:
i) a housing, which is provided with a support section for supporting a biochemical analysis unit within the housing, the biochemical analysis unit comprising a base plate, which has a plurality of holes, and a porous adsorptive material, which is filled in each of the plurality of the holes of the base plate and forms each of a plurality of adsorptive regions,
ii) a flow path, which is formed in the housing and enables a liquid to be circulated through each of the adsorptive regions of the biochemical analysis unit, and
iii) a flowing member, which is located in the flow path and circulates the liquid through the flow path by being actuated by actuating means.

The biochemical analysis unit structure body in accordance with the present invention should preferably be modified such that the flowing member is releasably connected to the actuating means, which is located on the side outward from the housing.

Also, the biochemical analysis unit structure body in accordance with the present invention should preferably be modified such that the housing is further provided with:
a liquid introducing port, through which the liquid is capable of being introduced into the housing, and
a flow path change-over member, the position of the flow path change-over member being capable of being changed over between a position for communication, at which the flow path change-over member allows the flow path to communicate with the liquid introducing port, and a position for circulation, at which the flow path change-over member blocks the flow path from the liquid introducing port and enables the liquid to be circulated through the flow path.

Further, the biochemical analysis unit structure body in accordance with the present invention should preferably be modified such that the flow path change-over member is releasably connected to actuating means, which is located on the side outward from the housing.

The liquid introducing port, through which the liquid is capable of being introduced into the housing, may also acts as a liquid discharging port, through which the liquid is capable of being discharged from the housing to the side outward from the housing. The liquid introducing port may thus constitute a liquid introducing and discharging port.

The biochemical analysis unit structure body in accordance with the present invention may also be modified such that a perforated plate having a plurality of micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of two surfaces of the biochemical analysis unit.

The biochemical analysis unit structure body in accordance with the present invention comprises the housing, which is provided with the support section for supporting the biochemical analysis unit within the housing. The biochemical analysis unit structure body in accordance with the present invention also comprises the flow path, which is formed in the housing and enables the liquid to be circulated through each of the adsorptive regions of the biochemical analysis unit. The biochemical analysis unit structure body in accordance with the present invention further comprises the flowing member, which is located in the flow path and circulates the liquid through the flow path by being actuated by the actuating means. Therefore, with the biochemical analysis unit structure body in accordance with the present invention, the volume within the structure body is capable of being kept smaller than the volume within a biochemical analysis reactor, wherein a flow path is connected to external flowing means, which comprises actuating means and a flowing member, and wherein a liquid is circulated by the external flowing means through the flow path. Accordingly, with the biochemical analysis unit structure body in accordance with the present invention, in cases where the amount of the liquid is small, the liquid is capable of being caused to penetrate sufficiently and uniformly into the interior of each of the adsorptive regions of the biochemical analysis unit. Also, since a sample need not be diluted in order for the amount of the liquid to be increased, a reaction is capable of being performed in a state, in which the concentration of the sample in the liquid is high, and the reaction rate is thus capable of being kept high.

Further, in order for accurate data to be obtained from the biochemical analysis, it is important to prevent the problems with regard to contamination by foreign substances from occurring. With the biochemical analysis unit structure body in accordance with the present invention, wherein the flow path, through which the liquid that will cause contamination to occur is circulated, and the flowing member for circulating the liquid through the flow path are located within the housing, instead of time and labor being required to wash and sterilize the structure body after each analysis has been made with the structure body, the structure body having been used for the analysis may be scrapped, and contamination is thus capable of being easily prevented from occurring. In particular, with the biochemical analysis unit structure body in accordance with the present invention, wherein external actuating means may be employed as the actuating means for actuating the flowing member, the actuating means itself does not come into contact with the liquid, which is circulated through the flow path. Therefore, the advantage is capable of being obtained in that the actuating means itself, whose cost is comparatively high, is capable of being used repeatedly.

The biochemical analysis unit structure body in accordance with the present invention may be modified such that the perforated plate having the plurality of the micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of the two surfaces of the biochemical analysis unit. With the modification described above, the perforated plate may be located on the side downstream from the biochemical analysis unit with respect to the direction of the flow of the liquid. In such cases, even though the liquid is forcibly circulated by the flowing member, and a liquid pressure is exerted on the biochemical analysis unit, the biochemical analysis unit is capable of being prevented from undergoing deflection and deformation.

Also, with the modification described above, the perforated plate may be located on the side upstream from the biochemical analysis unit with respect to the direction of the flow of the liquid. In such cases, by the effect of the micro-channels of the perforated plate, the flow rate of the liquid passing through each of the adsorptive regions of the biochemical analysis unit is capable of being enhanced. Therefore, the binding reaction rate is capable of being enhanced even further. Further, with the modification described above, in cases where the perforated plate is located so as to stand facing the two surfaces of the biochemical analysis unit, the biochemical analysis unit is capable of being prevented from undergoing deflection and deformation, and the binding reaction rate is capable of being enhanced even further.

### BRIEF DESCRIPTION OF THE DRAWINGS

FIG. 1 is a schematic sectional view showing a first embodiment of the biochemical analysis unit structure body in accordance with the present invention,

FIG. 2 is a schematic sectional view showing a second embodiment of the biochemical analysis unit structure body in accordance with the present invention,

FIG. 3 is a schematic sectional view showing a third embodiment of the biochemical analysis unit structure body in accordance with the present invention, and

FIG. 4 is a schematic sectional view showing a fourth embodiment of the biochemical analysis unit structure body in accordance with the present invention.

### DESCRIPTION OF THE PREFERRED EMBODIMENTS

The present invention will hereinbelow be described in further detail with reference to the accompanying drawings.

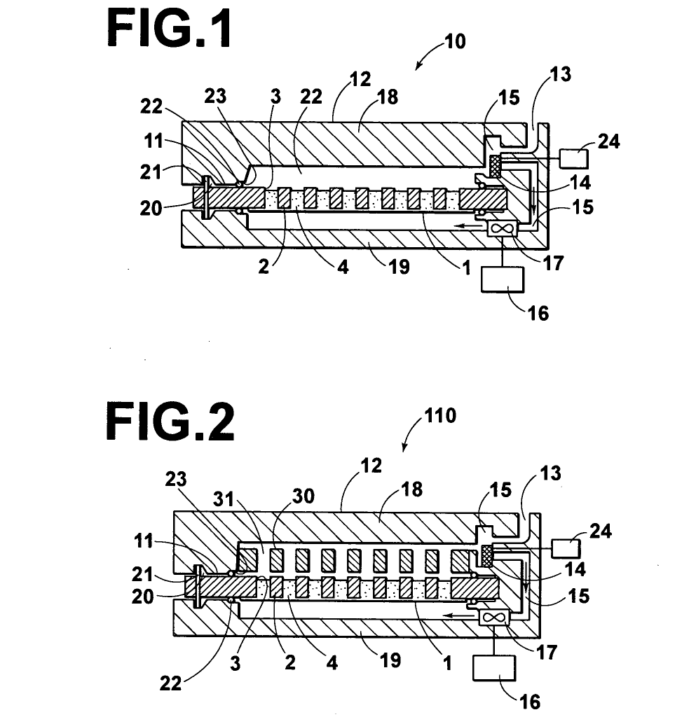

FIG. 1 is a schematic sectional view showing a first embodiment of the biochemical analysis unit structure body in accordance with the present invention. With reference to FIG. 1, a biochemical analysis unit structure body 10, which is the first embodiment of the biochemical analysis unit structure body in accordance with the present invention, comprises a housing 12, which is provided with a support section 11 for supporting a biochemical analysis unit 1 within the housing 12. The biochemical analysis unit structure body 10 also comprises a flow path 15, which is formed in the housing 12 and enables a liquid to be circulated through each of adsorptive regions 4, 4, . . . of the biochemical analysis unit 1. The biochemical analysis unit structure body 10 further comprises a flowing member 17, which is located in the flow path 15 and circulates the liquid through the flow path 15 by being actuated by an external motor 16, which acts as the actuating means. The biochemical analysis unit structure body 10 still further comprises a change-over valve 14, which acts as the flow path change-over member and is actuated by an external motor 24.

The biochemical analysis unit 1 comprises a base plate 2, which has a plurality of holes 3, 3, . . . The biochemical analysis unit 1 also comprises a porous adsorptive material, which is filled in each of the plurality of the holes 3, 3, . . . of the base plate 2 and forms each of a plurality of the adsorptive regions 4, 4, . . . .

The housing 12 is constituted of an upper housing half 18 and a lower housing half 19. The support section 11 for supporting the biochemical analysis unit 1 is provided with a depressed region 21 for receiving a position setting member 20, which adjusts the position of the biochemical analysis unit 1 when the biochemical analysis unit 1 is supported by the support section 11. The support section 11 is also provided with a sealing material receiving step-like section 23 for receiving a sealing material 22. When the biochemical analysis unit 1 is supported by the support section 11, the sealing material 22 sandwiches the peripheral areas of the base plate 2 of the biochemical analysis unit 1 and thus seals the biochemical analysis unit 1. The position of the biochemical analysis unit 1 is adjusted by the depressed region 21 and at a predetermined position within the lower housing half 19. Also, the peripheral areas of the base plate 2 of the biochemical analysis unit 1 are sandwiched by the sealing material 22, and the upper housing half 18 is set on the lower housing half 19. The biochemical analysis unit 1 is thus accommodated within the housing 12.

The housing 12 is provided with a liquid introducing and discharging port 13, which is connected to the flow path 15 and through which the liquid to be circulated through the flow path 15 is introduced into the housing 12 and discharged from the housing 12. The change-over valve 14 is located in the flow path 15. The position of the change-over valve 14 is capable of being changed over between a position for communication, at which the change-over valve 14 allows the flow path 15 to communicate with the liquid introducing and discharging port 13, and a position for circulation, at which the change-over valve 14 blocks the flow path 15 from the liquid introducing and discharging port 13 and enables the liquid to be circulated through the flow path 15. The change-over valve 14 is capable of being connected to the external motor 24, which is located on the side outward from the biochemical analysis unit structure body 10. Also, the flowing member 17, which is located in the flow path 15, is capable of being connected to the external motor 16, which is located on the side outward from the biochemical analysis unit structure body 10.

The biochemical analysis unit 1 is supported by the support section 11 and accommodated within the housing 12. Also, the liquid to be forcibly circulated through the flow path 15 is introduced through the liquid introducing and discharging port 13 into the housing 12. The liquid to be forcibly circulated through the flow path 15 may be a reaction liquid, which contains a substance to be subjected to a reaction with the adsorptive regions 4, 4, . . . of the biochemical analysis unit 1. Alternatively, the liquid to be forcibly circulated through the flow path 15 may be, for example, a washing liquid for washing the adsorptive regions 4, 4, . . . of the biochemical analysis unit 1. As another alternative, the liquid to be forcibly circulated through the flow path 15 may be a liquid containing a blocking agent for blocking the adsorptive regions 4, 4, . . . of the biochemical analysis unit 1. As a further alternative, the liquid to be forcibly circulated through the flow path 15 may be one of other kinds of liquids.

After the liquid has been introduced through the liquid introducing and discharging port 13 into the housing 12, such that the flow path 15 forms a circulation system, the external motor 24 is actuated in order to change over the position of the change-over valve 14 from the position for communication, at which the change-over valve 14 allows the flow path 15 to communicate with the liquid introducing and discharging port 13, to the position for circulation, at which the change-over valve 14 blocks the flow path 15 from the liquid introducing and discharging port 13 and enables the liquid to be circulated through the flow path 15. (The position of the change-over valve 14 illustrated in FIG. 1 is the position for communication. The position of the change-over valve 14 after being changed over upwardly in FIG. 1 is the position for circulation.) The liquid introducing and discharging port 13 is thus blocked by the change-over valve 14 having been set at the position for circulation. Also, the flowing member 17 of the biochemical analysis unit structure body 10 is connected to the external motor 16 and actuated by the external motor 16 in order to circulate the liquid, which has been introduced into the flow path 15, in the direction indicated by the arrow in FIG. 1.

The biochemical analysis unit structure body 10 is constructed such that the biochemical analysis unit 1 is accommodated within the housing 12. By way of example, the biochemical analysis unit 1 may have a 9 mm-square size and may be provided with 100 holes 3, 3, . . . , each of which has a hole diameter of 0.3 mm. In such cases, the proportion of the adsorptive regions 4, 4, . . . with respect to the biochemical analysis unit 1 is equal to 35%. Therefore, in such cases, the volume within a structure body, which is provided with a perforated plate, is 35% of the volume within the structure body, which is not provided with the perforated plate. Also, since the flow path 15, through which the liquid is circulated, and the flowing member 17, which circulates the liquid by being actuated by the external motor 16, are located within the housing 12, the volume within the structure body is capable of being kept small.

Specifically, in the cases of a biochemical analysis reactor, wherein a flow path is connected to external flowing means, which comprises actuating means and a flowing member, and wherein a liquid is circulated by the external flowing means through the flow path, it is necessary to use 1 ml of a reaction liquid. However, with the biochemical analysis unit structure body 10 in accordance with the present invention, a reaction is capable of being performed by use of 200 μl to 300 μl of the liquid. Therefore, with the biochemical analysis unit structure body 10 in accordance with the present invention, in which the volume within the structure body is small, in cases where the amount of a sample, which contains a receptor or a ligand to be analyzed with the biochemical analysis unit 1, (i.e., the amount of the reaction liquid) is small, the reaction liquid is capable of being caused to penetrate sufficiently and uniformly into the interior of each of the adsorptive regions 4, 4, . . . of the biochemical analysis unit 1. Also, since the sample need not be diluted in order for the amount of the reaction liquid to be increased, the reaction is capable of being performed in a state, in which the concentration of the sample in the reaction liquid is high, and the reaction rate is thus capable of being kept high.

Further, with the biochemical analysis unit structure body 10 in accordance with the present invention, wherein the flow path 15, through which the liquid is circulated, and the flowing member 17, which circulates the liquid through the flow path 15, are located within the housing 12, instead of time and labor being required to wash and sterilize the structure body after each analysis has been made with the structure body, the biochemical analysis unit structure body 10 having been used for the analysis may be scrapped, and contamination is thus capable of being easily prevented from occurring. Furthermore, the external motor 16, which acts as the actuating means for actuating the flowing member 17 of the biochemical analysis unit structure body 10, and the external motor 24, which acts as the actuating means for actuating the change-over valve 14, do not come into contact with the liquid, which is circulated through the flow path 15. Therefore, the external motor 16 and the external motor 24, whose costs are comparatively high, are capable of being used repeatedly.

A second embodiment of the biochemical analysis unit structure body in accordance with the present invention will be described hereinbelow with reference to FIG. 2.

FIG. 2 is a schematic sectional view showing a second embodiment of the biochemical analysis unit structure body in accordance with the present invention. With reference to FIG. 2, a biochemical analysis unit structure body 110, which is the second embodiment of the biochemical analysis unit structure body in accordance with the present invention, comprises a perforated plate 30 having a plurality of micro-channels 31, 31, . . . The micro-channels 31, 31, . . . have been formed such that each of the micro-channels 31, 31, . . . corresponds to one of the plurality of the holes 3, 3, . . . of the base plate 2 of the biochemical analysis unit 1 and is capable of communicating with one of the plurality of the holes 3, 3, . . . of the base plate 2. The perforated plate 30 is accommodated within the housing 12 such that the perforated plate 30 is located on the side downstream from the biochemical analysis unit 1 with respect to the direction of flow of the liquid.

The perforated plate 30 has the plurality of the micro-channels 31, 31, . . . , which have been formed such that each of the micro-channels 31, 31, . . . corresponds to one of the plurality of the holes 3, 3, . . . of the base plate 2 of the biochemical analysis unit 1 and is capable of communicating with one of the plurality of the holes 3, 3, . . . of the base plate 2. The perforated plate 30 is secured to the upper housing half 18. In FIG. 2, the perforated plate 30 is formed such that each of the micro-channels 31, 31, corresponds to one of the plurality of the holes 3, 3, . . . of the base plate 2 of the biochemical analysis unit 1 and is capable of communicating with one of the plurality of the holes 3, 3, . . . of the base plate 2. Alternatively, a perforated plate may be formed such that several micro-channels communicate with one of the plurality of the holes 3, 3, . . . of the base plate 2 of the biochemical analysis unit 1.

The biochemical analysis unit structure body 110, which is the second embodiment of the biochemical analysis unit structure body in accordance with the present invention, is constituted in the same manner as that for the biochemical analysis unit structure body 10, which is the first embodiment of the biochemical analysis unit structure body in accordance with the present invention, except that the biochemical analysis unit structure body 110 is also provided with the perforated plate 30. In FIG. 2, similar elements are numbered with the same reference numerals with respect to FIG. 1.

In cases where the perforated plate 30 is accommodated within the housing 12, the volume within the biochemical analysis unit structure body is capable of being reduced even further. Therefore, as described above, in cases where the amount of the sample, which contains the receptor or the ligand to be analyzed with the biochemical analysis unit 1, is small, the reaction liquid is capable of being caused to penetrate sufficiently and uniformly into the interior of each of the adsorptive regions 4, 4, . . . of the biochemical analysis unit 1.

Also, in the biochemical analysis unit structure body 110, the liquid within the flow path 15 is circulated in the direction indicated by the arrow in FIG. 2 by the flowing member 17, which is located in the flow path 15, and the perforated plate 30 is located on the side downstream from the biochemical analysis unit 1 with respect to the direction of flow of the liquid. Therefore, in cases where the liquid is forcibly circulated, and a liquid pressure is exerted upon the biochemical analysis unit 1, the biochemical analysis unit 1 is capable of being held by the perforated plate 30 and thus prevented from undergoing deflection and deformation.

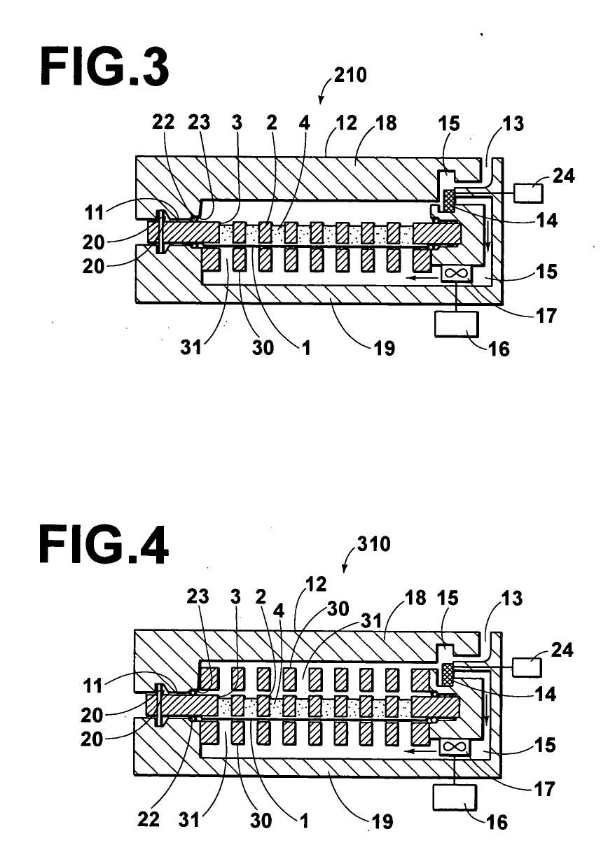

A third embodiment of the biochemical analysis unit structure body in accordance with the present invention will be described hereinbelow with reference to FIG. 3.

FIG. 3 is a schematic sectional view showing a third embodiment of the biochemical analysis unit structure body in accordance with the present invention. With reference to FIG. 3, a biochemical analysis unit structure body 210, which is the third embodiment of the biochemical analysis unit structure body in accordance with the present invention, comprises the perforated plate 30 having the plurality of the micro-channels 31, 31, . . . The micro-channels 31, 31, . . . have been formed such that each of the micro-channels 31, 31, . . . corresponds to one of the plurality of the holes 3, 3, . . . of the base plate 2 of the biochemical analysis unit 1 and is capable of communicating with one of the plurality of the holes 3, 3, . . . of the base plate 2. In the third embodiment, the perforated plate 30 is accommodated within the housing 12 such that the perforated plate 30 is located on the side upstream from the biochemical analysis unit 1 with respect to the direction of flow of the liquid.

The biochemical analysis unit structure body 210, which is the third embodiment of the biochemical analysis unit structure body in accordance with the present invention, is constituted in the same manner as that for the biochemical analysis unit structure body 110, which is the second embodiment of the biochemical analysis unit structure body in accordance with the present invention, except that the perforated plate 30 is located on the side upstream from the biochemical analysis unit 1 with respect to the direction of flow of the liquid. In FIG. 3, similar elements are numbered with the same reference numerals with respect to FIG. 2.

In cases where the perforated plate 30 is thus located on the side upstream from the biochemical analysis unit 1 with respect to the direction of flow of the liquid, the volume within the biochemical analysis unit structure body is capable of being reduced even further, and the flow rate of the liquid passing through each of the adsorptive regions 4, 4, . . . of the base plate 2 of the biochemical analysis unit 1 is capable of being enhanced by the effect of the micro-channels 31, 31, . . . of the perforated plate 30. Therefore, the binding reaction rate is capable of being enhanced even further.

A fourth embodiment of the biochemical analysis unit structure body in accordance with the present invention will be described hereinbelow with reference to FIG. 4.

FIG. 4 is a schematic sectional view showing a fourth embodiment of the biochemical analysis unit structure body in accordance with the present invention. With reference to FIG. 4, a biochemical analysis unit structure body 310, which is the fourth embodiment of the biochemical analysis unit structure body in accordance with the present invention, comprises two perforated plates 30, 30, each of which has the plurality of the micro-channels 31, 31, . . . The micro-channels 31, 31, . . . have been formed such that each of the micro-channels 31, 31, . . . corresponds to one of the plurality of the holes 3, 3, . . . of the base plate 2 of the biochemical analysis unit 1 and is capable of communicating with one of the plurality of the holes 3, 3, . . . of the base plate 2. In the fourth embodiment, the perforated plates 30, 30 are accommodated within the housing 12 such that the perforated plates 30, 30 are located on the opposite sides of the biochemical analysis unit 1, respectively.

The biochemical analysis unit structure body 310, which is the fourth embodiment of the biochemical analysis unit structure body in accordance with the present invention, is constituted in the same manner as that for the biochemical analysis unit structure body 210, which is the third embodiment of the biochemical analysis unit structure body in accordance with the present invention, except that the two perforated plates 30, 30 are located on the opposite sides of the biochemical analysis unit 1, respectively. In FIG. 4, similar elements are numbered with the same reference numerals with respect to FIG. 3.

In cases where the two perforated plates 30, 30 are thus located on the opposite sides of the biochemical analysis unit 1, respectively, the problems are capable of being efficiently prevented from occurring in that the biochemical analysis unit 1 undergoes deflection and deformation. Also, the volume within the structure body is capable of being reduced even further. Further, the flow rate of the liquid passing through each of the adsorptive regions 4, 4, . . . of the base plate 2 of the biochemical analysis unit 1 is capable of being enhanced, and the binding reaction rate is capable of being enhanced even further.

Examples of materials for the perforated plate 30 include metals, such as copper, silver, gold, zinc, lead, aluminum, titanium, tin, chromium, iron, and nickel; alloys, such as stainless steel and bronze; ceramic materials, such as alumina and zirconia; polyolefins, such as a polyethylene and a polypropylene; polystyrenes; acrylic resins, such as a polymethyl methacrylate; polyvinyl chlorides; polyvinylidene chlorides; polyvinylidene fluorides; polytetrafluoroethylenes; polychlorotrifluoroethylenes; polycarbonates; and polyesters, such as a polyethylene naphthalate and a polyethylene terephthalate.

Perforation of the plurality of the micro-channels 31, 31, . . . through the perforated plate 30 may be performed with, for example, a punching technique for punching with a pin, a technique for electrical discharge machining, in which a pulsed high voltage is applied across electrodes in order to volatilize the perforated plate material, or a laser beam irradiation technique, in which a laser beam is irradiated to the perforated plate material in order to volatilize the perforated plate material. In cases where the material of the perforated plate 30 is a metal, the perforation of the plurality of the micro-channels 31, 31, . . . through the perforated plate 30 may be performed with an etching technique. In cases where the material of the perforated plate 30 is a plastic material, the perforation of the plurality of the micro-channels 31, 31, . . . through the perforated plate 30 may be performed with a molding technique.

The micro-channels 31, 31, . . . of the perforated plate 30 are formed such that each of the micro-channels 31, 31, . . . corresponds to one of the plurality of the holes 3, 3, . . . of the base plate 2 of the biochemical analysis unit 1. Ordinarily, the pitch of the holes 3, 3, . . . of the biochemical analysis unit 1 (i.e., the distance between the center points of two holes which are adjacent to each other) may fall within the range of 0.05 mm to 3 mm. Also, the spacing between two adjacent holes 3, 3 (i.e., the shortest distance between edges of two adjacent holes 3, 3) may ordinarily fall within the range of 0.01 mm to 1.5 mm. The number (the array density) of the holes 3, 3, . . . may ordinarily fall within the range of least 10 holes/cm2 to at least 1,000 holes/cm2. Therefore, the micro-channels 31, 31, . . . of the perforated plate 30 are formed such that each of the micro-channels 31, 31, . . . corresponds to one of the plurality of the holes 3, 3, . . . of the base plate 2 of the biochemical analysis unit 1, which is to be used in the biochemical analysis unit structure body.

The biochemical analysis unit structure body in accordance with the present invention is applicable broadly to various assay processes for:
i) obtaining a biochemical analysis unit provided with a plurality of porous adsorptive regions, to which ligands or receptors have been bound respectively,
ii) subjecting a reaction liquid containing at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iii) detecting the receptor or the ligand, which has thus been specifically bound to at least one of the ligands or at least one of the receptors, by the utilization of a labeling substance.

In a first aspect, the biochemical analysis unit structure body in accordance with the present invention is applicable to an assay process for:
i) obtaining a biochemical analysis unit provided with a plurality of porous adsorptive regions, to which ligands or receptors have been bound respectively,
ii) subjecting a reaction liquid containing at least one kind of a labeled receptor or at least one kind of a labeled ligand, which has been labeled with a labeling substance, to specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iii) detecting the labeled receptor or the labeled ligand, which has thus been specifically bound to at least one of the ligands or at least one of the receptors.

In such cases, the ligands or the receptors, which have been bound respectively to the porous adsorptive regions of the biochemical analysis unit, are the substances, whose characteristics, compositions, structures, base sequences, base lengths, and the like, are known. Examples of the ligands or the receptors include hormones, tumor markers, enzymes, antibodies, antigens, abzymes, other proteins, nucleic acids, cDNA's, DNA's, and RNA's.

Also, in such cases, the labeled receptor or the labeled ligand is the substance, which has been sampled from an organism through extraction, isolation, or the like, or has been subjected to chemical treatment after being sampled, and which has been labeled with the labeling substance. The labeled receptor or the labeled ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the labeled receptors or the labeled ligands include hormones, tumor markers, enzymes, antibodies, antigens, abzymes, other proteins, nucleic acids, DNA's, and mRNA's.

Examples of the labeling substances include a radioactive labeling substance, a fluorescent labeling substance, and a labeling substance capable of causing a chemical luminescence substrate to produce the chemical luminescence when being brought into contact with the chemical luminescence substrate. The labeling substance may be a substance, which is capable of producing radiation by itself, a substance, which is capable of emitting light by itself, a substance, which is capable of forming a color by itself, or a substance, which is capable of producing fluorescence by itself when being exposed to light. Alternatively, the labeling substance may be a substance, which is capable of causing a chemical substance to emit light, to form a color, or to produce the fluorescence through, for example, decomposition or reaction of the chemical substance when being brought into contact with the chemical substance. As for the former type of the labeling substance, a radioactive isotope may be employed as the radiation producing labeling substance. Also, an acridinium ester, or the like, may be employed as the light emitting labeling substance. Further, gold colloidal particles, or the like, may be employed as the color forming labeling substance. Furthermore, fluorescein, or the like, may be employed as the fluorescent labeling substance. As the latter type of the labeling substance, an enzyme may be employed. Examples of the enzymes include alkaline phosphatase, peroxidase, luciferase, and β-galactosidase. When one of the above-enumerated enzymes acting as the labeling substance is brought into contact with a chemical luminescence substrate, a dye substrate, or a fluorescence substrate, the enzyme is capable of causing the chemical luminescence substrate to produce the chemical luminescence, causing the dye substrate to form a color, or causing the fluorescence substrate to produce the fluorescence.

By way of example, in cases where the enzyme is alkaline phosphatase, peroxidase, or luciferase, the chemical luminescence substrate may be dioxetane, luminol, or luciferin, respectively. In cases where the enzyme is alkaline phosphatase, the dye substrate may be p-nitrophenyl phosphate. In cases where the enzyme is β-galactosidase, the dye substrate may be p-nitrophenyl-β-D-galactoside, or the like. In cases where the enzyme is alkaline phosphatase, the fluorescence substrate may be 4-methylumbelliferphosphoric acid. In cases where the enzyme is peroxidase, the fluorescence substrate may be 3-(4-hydroxyphenyl)-propionic acid. In cases where the enzyme is β-galactosidase, the fluorescence substrate maybe 4-methylumbellifer-β-D-galactoside, or the like.

In a second aspect, the biochemical analysis unit structure body in accordance with the present invention is applicable to an assay process for:
i) obtaining a biochemical analysis unit provided with a plurality of porous adsorptive regions, to which ligands or receptors have been bound respectively,
ii) subjecting a reaction liquid containing at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting a labeled body, which has been labeled with a labeling substance, to specific binding with the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the receptor or the ligand, which has been specifically bound to at least one of the ligands or at least one of the receptors.

The aforesaid second aspect of the assay process is the so-called sandwich technique, wherein the receptor or the ligand, which is to be detected, is sandwiched between the ligand or the receptor, which has been bound to the adsorptive region, and the labeled body. In this case, the receptor or the ligand, which is to be detected, is the substance, which has been sampled from an organism through extraction, isolation, or the like, or has been subjected to chemical treatment after being sampled, and which has been labeled with the labeling substance. The receptor or the ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the receptors or the ligands, which are to be detected, include hormones, tumor markers, enzymes, antibodies, antigens, abzymes, other proteins, nucleic acids, DNA's, and mRNA's.

The labeled body, which has been labeled with the labeling substance, is a body, which has been labeled with the labeling substance described above and is capable of undergoing the specific binding with a reaction site of the receptor or the ligand, which is to be detected. Examples of the labeled bodies include antigens, antibodies, hormones, tumor markers, enzymes, abzymes, other proteins, nucleic acids, cDNA's, DNA's, and RNA's, whose characteristics, compositions, structures, base sequences, base lengths, and the like, are known.

In a third aspect, the biochemical analysis unit structure body in accordance with the present invention is applicable to an assay process for:
i) obtaining a biochemical analysis unit provided with a plurality of porous adsorptive regions, to which ligands or receptors have been bound respectively,
ii) subjecting a reaction liquid containing at least one kind of an auxiliary substance-bound receptor or at least one kind of an auxiliary substance-bound ligand, to which an auxiliary substance has been bound, to specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting an auxiliary substance-combinable labeling substance, which is capable of undergoing specific binding with the auxiliary substance, to specific binding with the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the auxiliary substance-bound receptor or the auxiliary substance-bound ligand, which has been specifically bound to at least one of the ligands or at least one of the receptors.

The auxiliary substance is a substance capable of undergoing the binding with the auxiliary substance-combinable labeling substance. Examples of preferable auxiliary substances include antigens, such as digoxigenin, biotin, avidin, and fluorescein, and antibodies with respect to the above-enumerated antigens. Also, the auxiliary substance may be a biological binding partner, such as avidin with respect to biotin. In this case, the auxiliary substance-combinable labeling substance is a substance, which is capable of undergoing the specific binding with the auxiliary substance and has been labeled with the labeling substance described above.

How a biochemical analysis using the biochemical analysis unit structure body in accordance with the present invention is performed will be described hereinbelow by taking a chemical luminescence technique as an example and with reference to FIG. 2 showing the biochemical analysis unit structure body 110, which is the second embodiment of the biochemical analysis unit structure body in accordance with the present invention.

Firstly, the biochemical analysis unit 1 is taken out from the biochemical analysis unit structure body 110, and the ligands or the receptors are spotted respectively onto the adsorptive regions 4, 4, . . . of the biochemical analysis unit 1. Thereafter, the ligands or the receptors, which have thus been spotted respectively onto the adsorptive regions 4, 4, . . . the biochemical analysis unit 1, are fixed to the adsorptive regions 4, 4, . . . with ultraviolet light irradiation, or the like. After the ligands or the receptors have thus been fixed respectively to the adsorptive regions 4, 4, . . . the biochemical analysis unit 1, the biochemical analysis unit 1 is accommodated within the biochemical analysis unit structure body 110. Specifically, the upper housing half 18 of the biochemical analysis unit structure body 110 is detached from the lower housing half 19. Also, the fitting position of the biochemical analysis unit 1 is adjusted by position setting member 20. Further, the peripheral areas of the base plate 2 of the biochemical analysis unit 1 are sandwiched by the sealing material 22, and the upper housing half 18 is set on the lower housing half 19. The biochemical analysis unit 1 is thus accommodated within the housing 12.

In this example, the biochemical analysis unit structure body 110, in which the biochemical analysis unit 1 has already been set, is employed. Alternatively, the biochemical analysis unit 1 may be prepared, such that each of the plurality of the holes 3, 3, . . . of the base plate 2 of the biochemical analysis unit 1 is capable of communicating with one of the micro-channels 31, 31, . . . of the perforated plate 30, which is accommodated within the biochemical analysis unit structure body 110.

Thereafter, the flowing member 17 of the biochemical analysis unit structure body 110 is connected to the external motor 16. Also, the reaction liquid containing at least one kind of the labeled receptor or at least one kind of the labeled ligand is introduced through the liquid introducing and discharging port 13 into the housing 12. The external motor 24 is then actuated in order to change over the position of the change-over valve 14 from the position for communication, at which the change-over valve 14 allows the flow path 15 to communicate with the liquid introducing and discharging port 13, to the position for circulation, at which the change-over valve 14 blocks the flow path 15 from the liquid introducing and discharging port 13 and enables the liquid to be circulated through the flow path 15. A switch of the external motor 16 is then turned on in order to actuate the flowing member 17, and the reaction liquid is caused to flow in the direction indicated by the arrow in FIG. 2. The reaction liquid flows into each of the adsorptive regions 4, 4, . . . of the biochemical analysis unit 1 and passes through each of the micro-channels 31, 31, . . . of the perforated plate 30, which micro-channels communicate respectively with the adsorptive regions 4, 4, . . . of the biochemical analysis unit 1. The reaction liquid is thus circulated through the flow path 15. In this manner, the labeled receptor or the labeled ligand, which is contained in the reaction liquid, is subjected to the specific binding with the ligands or the receptors, each of which has been bound to one of the adsorptive regions 4, 4, . . . of the biochemical analysis unit 1.

With the biochemical analysis unit structure body 110, the flow path 15, through which the liquid is circulated, and the flowing member 17, which is located in the flow path 15, are located within the housing 12. Therefore, the volume within the structure body is capable of being kept small. Also, since the biochemical analysis unit 1 and the perforated plate 30 are accommodated within the housing 12, the volume within the structure body is capable of being reduced even further. Therefore, in cases where the amount of the reaction liquid containing the labeled receptor or the labeled ligand is small, the reaction liquid is capable of being caused to penetrate sufficiently and uniformly into the interior of each of the adsorptive regions 4, 4, . . . of the biochemical analysis unit 1. Further, since the sample need not be diluted in order for the amount of the reaction liquid to be increased, the reaction is capable of being performed in a state, in which the concentration of the labeled receptor or the labeled ligand in the reaction liquid is high, and the reaction rate is thus capable of being kept high.

Furthermore, in cases where the reaction liquid within the flow path 15 is forcibly caused to flow in the direction indicated by the arrow in FIG. 2 by the flowing member 17, a liquid pressure is exerted upon the biochemical analysis unit 1 from below the biochemical analysis unit 1. However, with the biochemical analysis unit structure body 110, the perforated plate 30 having the micro-channels 31, 31, . . . is located on the side downstream from the biochemical analysis unit 1 with respect to the direction of flow of the liquid. Therefore, the biochemical analysis unit 1 is capable of being prevented from undergoing deflection and deformation.

In order for the labeled receptor or the labeled ligand, which has not been specifically bound to the ligands or the receptors having been bound respectively to the adsorptive regions 4, 4, . . . of the biochemical analysis unit 1, to be removed, the biochemical analysis unit 1 having been set within the biochemical analysis unit structure body 110 should preferably be washed with a technique, in which the flowing member 17 is actuated by the external motor 16, and a washing liquid is thus forcibly causes to flow across each of the adsorptive regions 4, 4, . . . In such cases, since the washing liquid is forcibly caused to flow across each of the adsorptive regions 4, 4, . . . , the labeled receptor or the labeled ligand, which has not been specifically bound to the ligands or the receptors having been bound respectively to the adsorptive regions 4, 4, . . . of the biochemical analysis unit 1, is capable of being peeled off and removed efficiently. Therefore, the washing efficiency is capable of being enhanced markedly. At the time of the washing operation described above, a liquid pressure is exerted upon the biochemical analysis unit 1 from below the biochemical analysis unit 1. However, with the biochemical analysis unit structure body 110, by the effect of the perforated plate 30, which is located on the side downstream from the biochemical analysis unit 1 with respect to the direction of flow of the reaction liquid, the biochemical analysis unit 1 is capable of being prevented from undergoing deflection and deformation.

Before an enzyme-labeled antibody, which will be described later, is subjected to the specific binding with the labeled receptor or the labeled ligand having been specifically bound to at least one of the ligands, each of which has been bound to one of the adsorptive regions 4, 4, . . . of the biochemical analysis unit 1, or at least one of the receptors, each of which has been bound to one of the adsorptive regions 4, 4, . . . of the biochemical analysis unit 1, the adsorptive regions 4, 4, . . . should preferably be blocked with a blocking process, wherein a blocking buffer with respect to the enzyme-labeled antibody is forcibly caused to flow such that the blocking buffer flows across each of the adsorptive regions 4, 4, . . . With the blocking process, the problems are capable of being prevented from occurring in that the enzyme-labeled antibody is directly bound to the adsorptive regions 4, 4, . . . of the biochemical analysis unit 1.

Thereafter, the liquid circulation is ceased. Also, the external motor 24 is actuated in order to change over the position of the change-over valve 14, such that the flow path 15 communicates with the liquid introducing and discharging port 13. The washing liquid is thus discharged through the liquid introducing and discharging port 13 from the housing 12. A reaction liquid, which contains the enzyme-labeled antibody, is then introduced through the liquid introducing and discharging port 13 into the housing 12. Thereafter, the external motor 24 is actuated in order to change over the position of the change-over valve 14, such that the liquid introducing and discharging port 13 is closed. Also, the flowing member 17 is actuated by the external motor 16, and the reaction liquid, which contains the enzyme-labeled antibody, is forcibly caused to flow such that the reaction liquid flows across each of the adsorptive regions 4, 4, . . . of the biochemical analysis unit 1. The enzyme-labeled antibody is thus subjected to the specific binding with the labeled receptor or the labeled ligand. The enzyme-labeled antibody is the antibody with respect to the labeling substance of the labeled receptor or the labeled ligand, which antibody has been labeled with an enzyme. (In cases where the labeling substance of the labeled receptor or the labeled ligand is an antibody, the enzyme-labeled antibody is the antigen with respect to the labeling substance of the labeled receptor or the labeled ligand, which antigen has been labeled with an enzyme.)

After the enzyme-labeled antibody has thus been subjected to the specific binding with the labeled receptor or the labeled ligand, the liquid circulation is ceased. Also, the position of the change-over valve 14 is changed over, such that the flow path 15 communicates with the liquid introducing and discharging port 13. A washing liquid is then introduced through the liquid introducing and discharging port 13 into the housing 12. Thereafter, the external motor 24 is actuated in order to change over the position of the change-over valve 14, such that the liquid introducing and discharging port 13 is closed. Also, the flowing member 17 is actuated by the external motor 16, and the washing liquid is forcibly caused to flow such that the washing liquid flows across each of the adsorptive regions 4, 4, . . . of the biochemical analysis unit 1. The enzyme-labeled antibody, which has not been bound to the labeled receptor or the labeled ligand, is thus removed from the adsorptive regions 4, 4, . . . of the biochemical analysis unit 1.

The liquid circulation is then ceased. Also, the external motor 24 is actuated in order to change over the position of the change-over valve 14 from the position for circulation, at which the change-over valve 14 blocks the flow path 15 from the liquid introducing and discharging port 13 and enables the liquid to be circulated through the flow path 15, to the position for communication, at which the change-over valve 14 allows the flow path 15 to communicate with the liquid introducing and discharging port 13. The washing liquid is then discharged through the liquid introducing and discharging port 13 from the housing 12. Also, a chemical luminescence substrate is introduced through the liquid introducing and discharging port 13 into the housing 12. Thereafter, the external motor 24 is actuated in order to change over the position of the change-over valve 14 from the position for communication to the position for circulation, and the liquid introducing and discharging port 13 is thus closed. Further, the flowing member 17 is actuated by the external motor 16, and the chemical luminescence substrate is brought into contact with the enzyme-labeled antibody, which has been specifically bound to the labeled receptor or the labeled ligand of the adsorptive regions 4, 4, . . .

The biochemical analysis unit 1 is then taken out from the biochemical analysis unit structure body 110. In cases where the chemical luminescence substrate and the enzyme are brought into contact with each other as described above, the chemical luminescence having wavelengths falling within the visible light wavelength range is produced. Therefore, the produced chemical luminescence is then detected photoelectrically from the adsorptive regions 4, 4, . . . , and the image data for a biochemical analysis is thus formed. In this manner, the labeled receptor or the labeled ligand is capable of being detected and analyzed.

The flow path 15, the micro-channels 31, 31, . . . of the perforated plate 30, the flowing member 17, and the like, are of fine constitutions. Therefore, from the view point of the time and the cost, it will not be advantageous to wash and sterilize the flow path 15, the micro-channels 31, 31, . . . of the perforated plate 30, the flowing member 17, and the like, completely. From the view point of the prevention of contamination, after the biochemical analysis unit structure body 110 has been disconnected from the external motor 16 and the external motor 24, the biochemical analysis unit structure body 110 having been used for the analysis should preferably be scrapped. Since the external motor 16 and the external motor 24 are capable of being used repeatedly, prevention of the contamination is capable of being achieved at a low cost in the manner described above.

As described above, The biochemical analysis unit structure body in accordance with the present invention comprises the housing, which is provided with the support section for supporting the biochemical analysis unit within the housing. The biochemical analysis unit structure body in accordance with the present invention also comprises the flow path, which is formed in the housing and enables the liquid to be circulated through each of the adsorptive regions of the biochemical analysis unit. The biochemical analysis unit structure body in accordance with the present invention further comprises the flowing member, which is located in the flow path and circulates the liquid through the flow path by being actuated by the actuating means. Therefore, with the biochemical analysis unit structure body in accordance with the present invention, the volume within the structure body is capable of being kept smaller than the volume within a biochemical analysis reactor, wherein a flow path is connected to external flowing means, and wherein a liquid is circulated by the external flowing means through the flow path. Accordingly, with the biochemical analysis unit structure body in accordance with the present invention, in cases where the amount of the liquid is small, the liquid is capable of being caused to penetrate sufficiently and uniformly into the interior of each of the adsorptive regions of the biochemical analysis unit. Also, since the biochemical analysis unit structure body in accordance with the present invention is a disposable structure body, the problems with regard to the contamination are capable of being prevented from occurring.

1. A biochemical analysis unit structure body, comprising:
i) a housing, which is provided with a support section for supporting a biochemical analysis unit within the housing, the biochemical analysis unit comprising a base plate, which has a plurality of holes, and a porous adsorptive material, which is filled in each of the plurality of the holes of the base plate and forms each of a plurality of adsorptive regions,
ii) a flow path, which is formed in the housing and enables a liquid to be circulated through each of the adsorptive regions of the biochemical analysis unit, and
iii) a flowing member, which is located in the flow path and circulates the liquid through the flow path by being actuated by actuating means.

2. A biochemical analysis unit structure body as defined in claim 1 wherein the flowing member is releasably connected to the actuating means, which is located on the side outward from the housing.

3. A biochemical analysis unit structure body as defined in claim 1 wherein the housing is further provided with:
a liquid introducing port, through which the liquid is capable of being introduced into the housing, and
a flow path change-over member, the position of the flow path change-over member being capable of being changed over between a position for communication, at which the flow path change-over member allows the flow path to communicate with the liquid introducing port, and a position for circulation, at which the flow path change-over member blocks the flow path from the liquid introducing port and enables the liquid to be circulated through the flow path.

4. A biochemical analysis unit structure body as defined in claim 2 wherein the housing is further provided with:
a liquid introducing port, through which the liquid is capable of being introduced into the housing, and
a flow path change-over member, the position of the flow path change-over member being capable of being changed over between a position for communication, at which the flow path change-over member allows the flow path to communicate with the liquid introducing port, and a position for circulation, at which the flow path change-over member blocks the flow path from the liquid introducing port and enables the liquid to be circulated through the flow path.

5. A biochemical analysis unit structure body as defined in claim 3 wherein the flow path change-over member is releasably connected to actuating means, which is located on the side outward from the housing.

6. A biochemical analysis unit structure body as defined in claim 4 wherein the flow path change-over member is releasably connected to actuating means, which is located on the side outward from the housing.

7. A biochemical analysis unit structure body as defined in claim 1 wherein a perforated plate having a plurality of micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of two surfaces of the biochemical analysis unit.

8. A biochemical analysis unit structure body as defined in claim 2 wherein a perforated plate having a plurality of micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of two surfaces of the biochemical analysis unit.

9. A biochemical analysis unit structure body as defined in claim 3 wherein a perforated plate having a plurality of micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of two surfaces of the biochemical analysis unit.

10. A biochemical analysis unit structure body as defined in claim 4 wherein a perforated plate having a plurality of micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of two surfaces of the biochemical analysis unit.

11. A biochemical analysis unit structure body as defined in claim 5 wherein a perforated plate having a plurality of micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of two surfaces of the biochemical analysis unit.

12. A biochemical analysis unit structure body as defined in claim 6 wherein a perforated plate having a plurality of micro-channels, which micro-channels have been formed such that at least one micro-channel corresponds to each of the plurality of the holes of the base plate of the biochemical analysis unit and is capable of communicating with each of the plurality of the holes of the base plate, is accommodated within the housing such that, at the time at which the biochemical analysis unit has been supported by the support section within the housing, the perforated plate stands facing at least either one of two surfaces of the biochemical analysis unit.

13. A biochemical analysis unit structure body as defined in claim 1 wherein the biochemical analysis unit structure body is adapted for use in an assay process for:
i) obtaining a biochemical analysis unit provided with a plurality of porous adsorptive regions, to which ligands or receptors have been bound respectively,
ii) subjecting a reaction liquid containing at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iii) detecting the receptor or the ligand, which has thus been specifically bound to at least one of the ligands or at least one of the receptors, by the utilization of a labeling substance.

14. A biochemical analysis unit structure body as defined in claim 1 wherein the biochemical analysis unit structure body is adapted for use in an assay process for:
i) obtaining a biochemical analysis unit provided with a plurality of porous adsorptive regions, to which ligands or receptors have been bound respectively,
ii) subjecting a reaction liquid containing at least one kind of a labeled receptor or at least one kind of a labeled ligand, which has been labeled with a labeling substance, to specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iii) detecting the labeled receptor or the labeled ligand, which has thus been specifically bound to at least one of the ligands or at least one of the receptors.

15. A biochemical analysis unit structure body as defined in claim 1 wherein the biochemical analysis unit structure body is adapted for use in an assay process for:
i) obtaining a biochemical analysis unit provided with a plurality of porous adsorptive regions, to which ligands or receptors have been bound respectively,
ii) subjecting a reaction liquid containing at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting a labeled body, which has been labeled with a labeling substance, to specific binding with the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the receptor or the ligand, which has been specifically bound to at least one of the ligands or at least one of the receptors.

16. A biochemical analysis unit structure body as defined in claim 1 wherein the biochemical analysis unit structure body is adapted for use in an assay process for:
i) obtaining a biochemical analysis unit provided with a plurality of porous adsorptive regions, to which ligands or receptors have been bound respectively,
ii) subjecting a reaction liquid containing at least one kind of an auxiliary substance-bound receptor or at least one kind of an auxiliary substance-bound ligand, to which an auxiliary substance has been bound, to specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting an auxiliary substance-combinable labeling substance, which is capable of undergoing specific binding with the auxiliary substance, to specific binding with the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the auxiliary substance-bound receptor or the auxiliary substance-bound ligand, which has been specifically bound to at least one of the ligands or at least one of the receptors.
